(19) United States
(12) Patent Application Publication
(10) Pub. No.: US 2024/0390587 A1
(43) Pub. Date: Nov. 28, 2024

(54) DEVICE FOR DRUG INJECTION

(71) Applicant: Claudio Esteban Ouwerkerk, Quilmes (AR)

(72) Inventor: Claudio Esteban Ouwerkerk, Quilmes (AR)

(21) Appl. No.: 18/322,667

(22) Filed: May 24, 2023

(51) Int. Cl. A61M 5/28; A61M 5/32

(52) U.S. Cl. CPC A61M 5/28; A61M 5/3204

(57) ABSTRACT

A device for injecting medicinal solutions and liquid drugs in general by Carpule type syringes or the like, wherein the drug is contained in a tube having a proximal end or upper end with a plunger actionable by means of the stem of the Carpule syringe or the like, and a distal end or lower end with a plug of pierceable elastomeric material with the needle end fixed in a needle-holder piece. The distal end or lower end of the tube and the needle-holder piece are linked in the form of a male/female coupling, where the male part is the distal end of the tube and the female part is defined in a rear section of the needle-holder piece. The inlet end of the needle passes tightly through the front section of the needle-holder.

### FIELD OF THE INVENTION

The present invention relates to a device for the injection of drugs, and more specifically, it consists of an inviolable, disposable and single-use device which ensures that the biochemical and biophysical properties of the drug to be injected in each patient are preserved, so that they are not altered or modified until their application.

### STATE OF THE ART

Currently, injectable drugs are provided in a small hermetically sealed glass bottle with a lid which upper side has a disc of pierceable material with the needle of a syringe to extract the drug and load it into the syringe. The sterile needles to be used are generally contained in a plastic laminar container from which they are extracted by cutting off one end of the container. Although needles are used only once and discarded immediately after injecting the drug into a patient, and although it is understood that the medical or nursing professional acts responsibly in this way, there is no evidence that allows us to be absolutely certain that this is always the case, i.e., that each needle is discarded definitively after being used in a patient, which is a problem that deserves to be solved.

### SUMMARY OF THE INVENTION

The device that is the object of the present invention has been developed in order to provide an efficient solution to the risks of improper reuse of needles that must be used only once, even though such risks may be low. This device consists of a tube containing the drug, or tube, and the corresponding needle, which is presented as a joined, inviolable and sterile assembly. The drug tube can be made of plastic or glass. The inlet end of the needle is fixed in a pass-through manner in a needle-holder piece or intermediate. These components are kept inseparably coupled and, therefore, they grant the device with characteristics that make it inviolable, ensuring that the tube, together with the needle, can be used only once in a patient and then be discarded.

Among the various advantages of this device, it is worth mentioning that it provides biosafety for each particular patient, since, once used, it leaves irreversible physical evidence of its use; its structural and functional design means that the needle comes into contact with the drug only when the professional requires its use, a condition which ensures that the drug's properties and sterilization are not altered until the moment it is injected into the patient. The device prevents the needle from being reused, and it also prevents the tube from being reused, thus ensuring the sterilization and biosafety of the solution to be injected; and it reduces both handling and the professional's working time, among other advantages.

The object of the present invention is, therefore, to provide a device for injecting medicinal solutions and liquid drugs in general by means of Carpule type syringes or the like, wherein the medicine is contained in a tube having a proximal end or upper end with a plunger actionable by means of the stem of the Carpule syringe or the like, and a distal end or lower end with a plug of pierceable elastomeric material with the needle end fixed in a needle-holder piece. The distal end or lower end of the tube and said needle-holder piece are linked in the form of a male/female coupling, where the male part is the distal end of the tube and the female part is defined in a rear section of the needle-holder piece. The inlet end of the needle passes tightly through the front section of the needle-holder.

The external face of the male distal end of the drug container tube and the internal face of the female section of the needle-holder piece present respective successions of complementary projections that determine an irreversible and inviolable unidirectional non-returnable coupling between said tube and the needle-holder piece. Between the front and rear sections of the needle holder, there is a step that defines a circumferential stop that pushes the needle holder against the distal end of the tube by means of the removable cap that covers the needle, for the coupling and perforation of the tube cap and the passage of the inlet end of the needle into the tube. The device is presented as an assembly defined by the drug tube, the needle holder prefixed on a first projection, with the corresponding needle covered by the protective cap, ready to be placed in the Carpule syringe, or similar, and then to be activated by pressing the cap towards the tube to pierce the tube cap, thus being able to inject the drug into the patient. Additionally, the device may have marks showing that the system has not been used.

### BRIEF DESCRIPTION OF THE DRAWINGS

For greater clarity and understanding of the present invention, said invention has been illustrated in several figures representing it according to preferred forms of realization, all by way of example, wherein:

FIG. 1 shows a divided view of the device, which is the object of the present invention, that shows the tube containing the drug to be applied, the needle holder with the corresponding needle and the cap used to cover the needle before and after its use;

FIG. 2 is a longitudinal cut view showing how the device is provided to be then placed in a Carpule syringe or similar, where the drug container tube, the needle holder at a first coupling point to the distal end of said tube, and the corresponding needle covered by a protective cap are observed;

FIG. 3 is the “A” detail indicated in FIG. 2, which shows the needle holder positioned at a first point of engagement prior to puncture of the drug container tube by with the inlet end of the needle;

FIG. 4 is the “A” detail shown in FIG. 2, which also shows the distal end of a Carpule syringe, or similar, in which the device is incorporated. The device has a needle holder positioned at a second point of engagement with the needle, piercing through the drug tube;

FIG. 5 shows an example of the application of the device in a Carpule type syringe; and

FIG. 6 shows a perspective front view of the device according to the present invention.

### DETAILED DESCRIPTION OF THE INVENTION

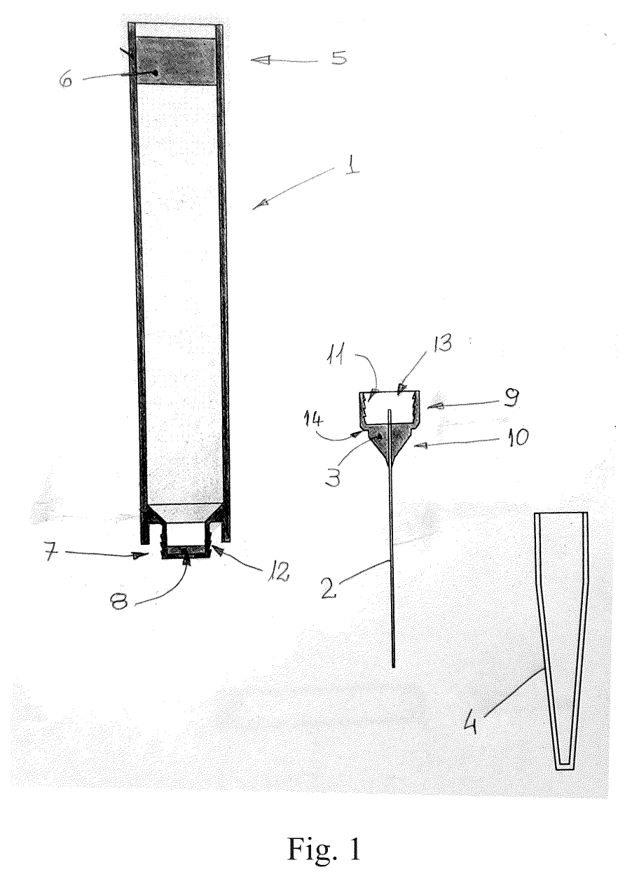

As illustrated in the divided view of FIG. 1, the device described is defined by an assembly comprising a tube 1 containing the drug to be injected into a patient, a needle 2, an intermediate or needle holder 3, and a cap 4 that serves the dual function of covering the needle 2 and serving as a push and coupling element of the needle holder 3 in the tube 1 in order to insert the upper end of needle 2 into the interior of said tube 1.

The proximal section 5 of the tube 1 contains the corresponding plunger 6 operable by means of the stem of the Carpule type syringe or similar. The distal end 7 contains a plug 8 of elastomeric material pierceable by means of the inlet end of the needle 2.

The needle holder 3 is linked to the tube 1 in the form of a male/female coupling, where the male part is the distal end 7 of said tube 1 and the female part is the cavity defined in the upper portion 9 of the needle holder 3, and the upper section of the needle 3 ending at the drug inlet end of the needle is tightly fixed by the lower portion 10 of the needle holder 3.

In order to achieve a male/female coupling between the needle holder 3 and the tube 1 in a unidirectional non-returnable, irreversible and inviolable manner, the external face of the male distal end 7 of the tube 1 and the internal face of the female section 9 of the needle holder 3 present, respectively, successions of complementary projections 11 and 12. As shown, said projections 11 and 12 are in the shape of a sawtooth, or similar, or they may be similar functionally equivalent projections, each of which, in this exemplary embodiment, has a longitudinal side inclined in the direction of the axial coupling of the needle holder 9 at the distal end 7 of the tube 1, and a side essentially transverse to the surfaces of the external face of said distal end 7 of the tube 1 and the internal face of the cavity 13 defined in the female section 9 of the needle holder 3. Since the inclination of the longitudinal sides of the saw teeth of the distal end 7 of the tube 1 is opposite to the longitudinal side of the corresponding saw teeth of the needle-holder piece 3, the respective transverse sides of the saw teeth jointly define the locking and retaining stops of the needle holder 3 in the tube 1.

Between the sections 9 and 10 of the needle holder 3, there is a step 14 that defines a circumferential stop of the needle holder 3 on which the cap 4 is pressed towards the tube 1 for its coupling and fixation on the distal end 7 of the tube 1.

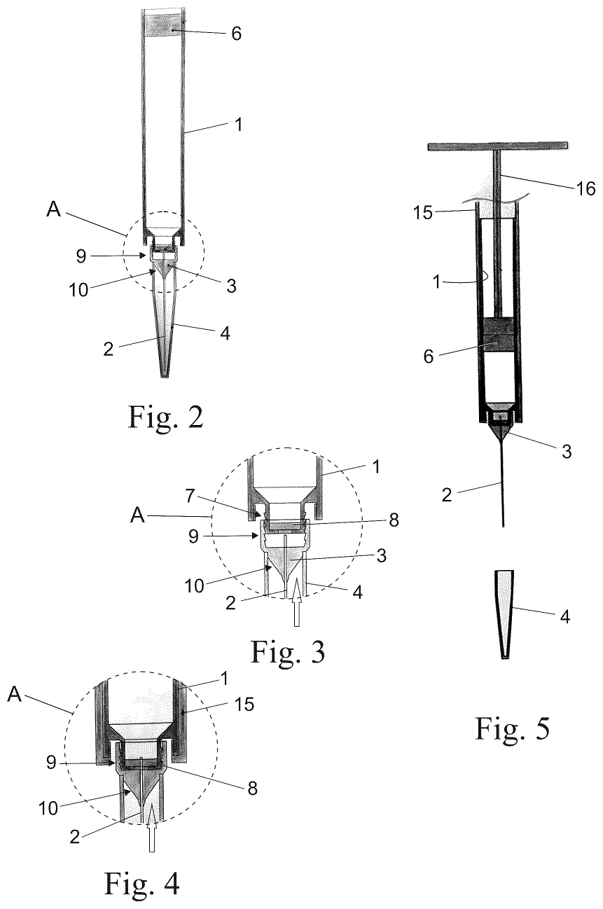

The device is provided with the needle holder 3 partially coupled to the tube 1 up to a first point of engagement or “click”, the inlet end of the needle 2 remaining in the vicinity of the plug 8, i.e., without piercing it, as shown in FIG. 2 and more clearly in the A detail of FIG. 3. Once the device has been placed in the tube 15 of a Carpule syringe, or similar, in order to proceed to the injection of the drug into a patient, the professional in charge must exert sufficient thrust force on the cap 4 so that the needle holder 3 reaches a second point of engagement at the distal end 7 of the tube 1, thus piercing the cap 8 of the tube 1 with the inlet end of the needle 2, which is thus inserted into the tube 1, and, finally, remove the cap 4, as illustrated in the FIG. 4. In other words, the assembly defined in this device by the drug-containing tube 1, the needle 2 and the needle holder 3 is activated by the pressure of the cap 4 on the circumferential stop 14 of the needle holder 3.

With respect to the coupling between the needle holder 3 and the tube 1, it should be mentioned that as the cap 4 is pressed against the tube 1, the successions of saw teeth 11 of the cavity 13 of the needle holder 3 are forced to move over the saw teeth of the distal end 7 of the tube 1. Once the coupling is completed, with the end of the needle 2 being inserted into the tube 1, the aforementioned transverse sides of the saw teeth successions face each other and, consequently, define a non-returnable coupling by blocking the backward movement of the needle holder 3, maintaining the coupling in an irreversible and inviolable manner.

Once the needle holder 3 is fully engaged in the tube 1 of the device, the cap 4 is removed and the Carpule syringe, or similar, with the tube 1 and needle 2, is ready to inject the drug into a patient. Once the drug injection has been completed and the needle has been removed from the patient, the needle 2 is again covered by the cap 4, as shown in FIG. 5. In practice, the cap 4 is preferably screwed onto the water-holder 3, but the coupling may also be, for example, of a “bayonet” type.

In the same FIG. 5, reference 16 shows the syringe stem acting on the plunger 6 of the tube 1 to push the drug into the needle 2 to inject the drug into the patient.

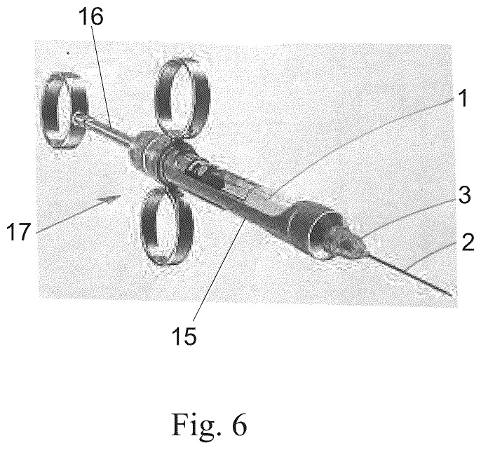

FIG. 6 illustrates, as an example of application, the device object of the present invention placed in a Carpule type syringe marked with reference number 17.

Thus, having especially described and determined the nature of the present invention and the manner in which it is to be brought into practice, it is hereby declared to claim as its exclusive property and right:

1. A device for injecting medicinal solutions and liquid drugs by carpule syringes or similar comprising:
an assembly defined by a tube (1) containing the liquid drug to be injected and a needle holder (3) with a needle (2) joined in the form of a unidirectional male coupling and a female coupling;
wherein the male coupling of the tube (1) is the distal end thereof and the female coupling is defined in an upper section (9) of the needle-holder piece (3);
wherein the tube (1) has a proximal section (5) in which a plunger (6) is arranged, which is actionable by a stem (16) of the syringe, and a distal end (7) where a plug (8) made of an elastomeric material, which can be pierced by the needle (2), is housed;
wherein an inlet section of the needle (2) is fixed in the needle holder (3) passing through a lower portion (10) thereof;
wherein an external face of the male distal end (7) of the tube (1) and an internal face of the upper female portion (9) of the needle-holder piece (3) present respective successions of complementary projections which determine the irreversible unidirectional coupling between said tube (1) and said needle-holder piece (3).

2. The device according to claim 1, wherein said projections are sawtooth-shaped and have a longitudinal side with respect to a direction of an axial coupling of the needle-holder piece with said tube, an inclination of the longitudinal side of the sawtooth-shaped of the distal end of the tube is oriented in an opposite direction to the longitudinal side of the corresponding sawtooth-shaped of the needle-holder piece.

3. The device according to claim 2, wherein the transverse sides of the sawtooth-shaped of the distal end of the tube and of the needle-holder piece together define non-returnable locking stops of the coupling between the tube and the needle holder.

4. The device according to claim 1, wherein between a front section and a rear section of the needle holder there is a perimeter step that defines a circumferential stop for pushing the needle holder by a needle cap against the distal end of the tube for the coupling and piercing of the needle cap with the needle and the passage of the needle inlet end into the tube.
